(19) United States
(12) Patent Application Publication
(10) Pub. No.: US 2026/0216217 A1
(43) Pub. Date: Jul. 30, 2026

(54) CANNABINOID SOLUBILIZATE

(71) Applicant: AQUANOVA AG, Darmstadt (DE)

(72) Inventor: Dariush BEHNAM, Rossdorf (DE)

(73) Assignee: AQUANOVA AG, Darmstadt (DE)

(21) Appl. No.: 19/146,997

(22) PCT Filed: Jan. 11, 2023
(86) PCT No.: PCT/EP2023/050523

§ 371 Date: Jul. 10, 2025

(51) Int. Cl. A61K 31/00; A61K 9/107; A61K 47/14; A61K 47/26

(52) U.S. Cl. CPC A61K 31/658; A61K 9/107; A61K 47/14; A61K 47/26

(57) ABSTRACT

In order to quickly provide the highest possible bioavailability of cannabinoids, which in particular are in the form of an isolate, with a stable formulation, the invention provides a solubilizate comprising at least one cannabinoid, in particular cannabinoid isolate, at least one emulsifier having an HLB value in the range of less than 18, preferably between 13 and 18, in particular polysorbate 80 or polysorbate 20, or at least one sugar ester of edible fatty acids, or a mixture of at least two emulsifiers, selected from the group comprising polysorbate 20, polysorbate 80 and sugar esters of edible fatty acids, and medium-chain triglycerides.

The invention relates to a solubilizate comprising cannabinoids and medium-chain triglyceride according to claim 1. Furthermore, the invention relates to a product containing such a solubilizate, to a capsule filled with such a solubilizate, and to a method for its preparation, and to the use of the solubilizate for being used in the treatment and/or prevention of sleep disorders (insomnia), depression and/or anxiety.

Unsatisfactory duration or quality of sleep impairs well-being and performance during the day and can lead to considerable suffering for those affected. Such a sleep disorder, also referred to as insomnia, is present if the symptoms occur three times a week for three months, or if shorter phases occur over several years. (See “International Classification of Sleep Disorders” ICSD-3). Those affected then in particular have the impression of insufficient sleep or do not feel refreshed after the usual sleep time.

The prevalence of insomnia in Germany is between 10 and 50%, with occasional insomnia affecting 25 to 30% of the total population and chronic insomnia affecting 10 to 13%. Chronic insomnia is associated with psychiatric disorders, among other things. For example, the risk of depression is increased by 2.6 times. The risk of myocardial infarction (heart attack) and apoplexy (stroke) is also increased, by up to 70%. Furthermore, affective disorders/bipolar disorders, anxiety disorders, panic disorders, post-traumatic stress disorder (PTSD), alcohol abuse (alcohol dependence), borderline disorders, dementia, eating disorders, and schizophrenia have been associated with sleep disorders (information as of Dec. 29, 2022 in http://www.gesundheits-lexikon.com/Schlaf-Schlafstoerungen/Schlafstoerungen-Insomnie/).

One current therapeutic approach for sleep disorders is cannabinoid use. Clinical interest in the therapeutic potential of cannabinoids in the treatment of sleep disorders is increasing. Growing evidence suggests that the endocannabinoid system plays a role in the regulation of the circadian sleep-wake cycle (see, e.g., Isobel Lavender et al. “Cannabinoids, Insomnia, and Other Sleep Disorders” in: Sleep: CHEST Reviews, Volume 162, ISSUE 2, P452-465, Aug. 1, 2022, https://doi.org/10.1016/j.chest.2022.04.151.).

Cannabinoids are transformation products and synthetic analogues of some terpene phenols found primarily in hemp plants (Cannabis). The terms “hemp plant” or “cannabis plant” includes the wild type Cannabis sativa and also variants thereof, including Cannabis chemovars, which naturally contain different amounts of the various cannabinoids, Cannabis sativa Subspecies indica, and also plants that are the result of genetic crosses, self-crosses or hybrids thereof.

The cannabis plant contains at least 113 cannabinoids from the terpene phenol group. Some of these cannabinoids are delta9-tetrahydrocannabinol (THC), delta8-tetrahydrocannabinol, cannabidiol (CBD), cannabichromene (CBC), cannabigerol (CBG), cannabinol (CN), delta9-tetrahydrocannabivarin (THCV), cannabicyclol (CBL), cannabielsoin (CBE), cannabitriol (CBT), and cannabinodiol (CBND).

Tetrahydrocannabinol (THC) is a psychoactive substance that is said to be mainly responsible for the intoxicating effect of cannabis products. In the plant, THC occurs naturally in the form of two THC acids, which are converted into THC when the plant material is heated. Cannabidiol (CBD) is a non-psychoactive cannabinoid which, among other things, also has antispasmodic, anxiolytic, and neuroprotective effects. Cannabichromene (CBC), like CBD, has no intoxicating effect. In particular, it is said to have an antidepressant effect. Cannabinol (CBN) is an oxidation product of THC and has an intoxicating effect as well as pain-relieving, antispasmodic, and calming effects. Cannabigerol (CBG) is not psychoactive and is said to have a greater pain-relieving effect than THC. Tetrahydrocannabivarin (THCV) has a lower psychoactive effect compared to THC and, among other things, has an appetite-suppressing and metabolism-stimulating effect.

Cannabis also contains a variety of non-cannabinoids with diverse pharmacological properties. There is evidence that cannabinoids such as cannabinol (CBN), cannabidiol (CBD), and others modify the effects of Δ9-THC.

Artificial cannabinoids can be produced both semi-synthetically, i.e. from natural cannabinoids, and fully synthetically, from simple basic substances. Other plants can also produce active substances that act in the human body via the same mechanisms as the cannabinoids of the hemp plant. For the purposes of the present application, the term “cannabinoid” shall encompass both natural and artificial cannabinoids.

In order to enter the bloodstream after oral administration, the active substance must pass through the small intestinal blood barrier, is then metabolized in the liver and released into the hepatic vein as a bioavailable fraction. The remainder of the total active substance ingested and released into the body is either broken down microbially in the intestine or eliminated with the feces or bile.

The inventor therefore set himself the task of providing a formulation which makes the calming and healing properties of cannabinoids, in particular CBD, with regard to sleep disorders (insomnia), depression and/or anxiety accessible to the human or animal organism in an easy-to-use manner.

In particular, it is an object of the invention to quickly enable the highest possible bioavailability of cannabinoids.

The inventor recognized that, in contrast to other plant extracts, the temporal distribution of the rate of absorption is of great importance for cannabinoids and CBD in particular. With optimized pharmacokinetics, the effects described above can then be achieved as quickly as possible and thus in a targeted manner instead of being undesirably delayed and/or over a longer period of time.

Cannabinoids are available in various forms. A distillate is a highly refined natural extract that has undergone a distillation process. For example, with a CBD content of typically 75% or more, a CBD distillate is highly concentrated. Other plant compounds such as flavonoids, terpenes, and cannabinoids (including THC) make up the remainder of the CBD distillate.

An isolate is technically the purest available form of an active substance, in this case a cannabinoid, and essentially does not include any other components of the source material. For example, CBD isolate is produced using a natural extraction process that uses CBD distillate.

This eliminates all the components that give hemp its flavor and aroma. The isolate therefore has no taste or smell. CBD isolate is a crystalline solid. A cannabinoid isolate therefore contains the maximum technically possible content of active substance in relation to its mass.

It is a further object of the invention to provide a stable formulation for cannabinoids, in particular for CBD, which are provided in the form of an isolate.

These objects are achieved in a surprisingly simple manner with a solubilizate according to claim 1 and a method for its preparation. This solubilizate consists of or comprises:
at least one cannabinoid, in particular a cannabinoid isolate, preferably CBD isolate,
at least one emulsifier having an HLB value in the range of less than 18, preferably between 13 and 18, in particular polysorbate 80 or polysorbate 20, or at least one sugar ester of edible fatty acids, or a mixture of at least two emulsifiers, selected from the group comprising polysorbate 20, polysorbate 80 and sugar esters of edible fatty acids, and
medium-chain triglycerides (MCT).

The components of the solubilizate add up to 100 wt %. If further optional components of the solubilizate according to the invention are mentioned below, this applies accordingly.

Medium-chain triglycerides (MCT) are triglycerides which comprise medium-chain fatty acids. Medium-chain fatty acids include caproic acid, caprylic acid, capric acid, and lauric acid. These are saturated fatty acids that occur naturally in tropical vegetable fats such as coconut oil and palm kernel oil. They are also present in small amounts in milk fat. There is no pure MCT oil in nature, but pure MCT oils can be obtained synthetically. Within the scope of the invention, individual MCTs or a mixture of different MCTs can be used as medium-chain triglycerides.

Surprisingly, it has been found that especially the composition made up of at least one cannabinoid, emulsifier and medium-chain triglycerides (MCT) according to the invention provides a stable formulation for cannabinoids, even if provided in the form of an isolate as the starting material. In the solubilizate according to the invention, the cannabinoid is enclosed in micelles. The micelles can be measured as particles, for example in an aqueous dilution of the solubilizate, using laser light diffraction.

In contrast to a formulation in the form of an emulsion, which is provided with the active substance dissolved in a liquid phase, in particular an oil, and drops thereof dispersed in another liquid, in particular an aqueous phase, the micellar structure of the solubilizate according to the invention is very stable. The invention thus provides a formulation of product micelles loaded with cannabinoid as the active substance, so that the active substance is protected from environmental influences and, due to the specific structure of the solubilizate, is highly bioavailable. Bioavailability will be discussed in more detail further below.

In this way a solubilizate is provided which is suitable for oral intake of such a dose of cannabinoids, in particular CBD, by humans or animals, so that the calming and healing properties thereof with regard to sleep disorders (insomnia), depression and/or anxiety states are made accessible to the human or animal organism. One reason for this is the comparatively short time in which a comparatively large amount of cannabinoid from the solubilizate according to the invention is absorbed by the organism. Reference for the comparison is a solution of the isolate in glycerol. This was demonstrated in an animal study, which will be discussed in more detail further below.

According to a preferred embodiment of the invention, the solubilizate comprises medium-chain triglycerides in a proportion of up to 10 wt %, preferably between 1 wt % and 8 wt %, most preferably up to 5 wt %.

According to a refinement of the invention, the solubilizate may contain, as a further component, up to 10 wt % of glycerol, preferably between 1 wt % and 8 wt %, most preferably up to 5 wt %.

Depending on the field of application, the solubilizate can be produced with at least one antioxidant within the scope of the invention. For this purpose, it is contemplated for the solubilizate to contain, as a further component, up to 5 wt % of at least one antioxidant, preferably between 0.5 wt % and 3 wt %, most preferably between 1 wt % and 2 wt %, preferably at least one tocopherol, most preferably mixed tocopherol.

It is preferred, within the scope of the invention, to use alpha-tocopherol and/or beta-tocopherol and/or gamma-tocopherol and/or delta-tocopherol or to use mixed tocopherols consisting of alpha-tocopherol, beta-tocopherol, gamma-tocopherol and delta-tocopherol. It has been found that the use of the same amount of mixed tocopherols gives the solubilizate according to the invention a greater antioxidant potential than the use of, for example, alpha-tocopherol alone. A mixture of alpha-tocopherol, beta-tocopherol, gamma-tocopherol and delta-tocopherol will be referred to as “mixed tocopherol” in the present description and the appended claims.

Chemically lipophilic antioxidants may be used in addition to or as an alternative to tocopherol within the scope of the invention. Examples include butylhydroxyanisole (BHA; E320), butylhydroxytoluene (BHT, E321), gallates (E310 to 312), or rosemary extract with the active substances carnosol and carnosic acid (E392). The so-called “E numbers” mentioned refer to the list of food additives approved by the European Union.

In order to provide stable micelles of the cannabinoid, the emulsifier content, in particular the polysorbate content, is at least 70 wt % within the scope of the invention, preferably in the range between 75 wt % and 98 wt %, most preferably in the range between 82 wt % and 93 wt %.

The particularly small size of the micelles in the solubilizate according to the invention results in a clear and permanently transparent product. The solubilizates according to the invention have a narrow particle size distribution with small average particle sizes, even under the physiological conditions of gastric passage. Preferably, the diameter distribution of the micelles in a dilution of the solubilizate with distilled water in a ratio of 1:500 at pH 1.1 and 37° C. ranges from about d₁₀=5 nm to about d₉₀=20 nm, particularly preferably from about d₁₀=6 nm to about d₉₀=13 nm. These values were determined on the basis of a volume distribution.

The particle size analysis of the micelles in an aqueous dilution of the solubilizate according to the invention was performed using the principle of dynamic light scattering with the ParticleMetrix NANOFLEX backscattering particle analyzer. Prior to dilution, the samples were heated to 37° C. in a water bath and redispersed by shaking and stirring. The samples were then diluted 1:500 with distilled water and heated to 37° C. with continuous stirring using a magnetic stirrer hotplate. The pH was then adjusted to approximately 1.1 with 32% HCl. The samples were then measured immediately.

An indication of the improved bioavailability of a cannabinoid compared to compositions not micellized according to the invention is provided by a determination of the turbidity of the solubilizate, which is significantly easier to accomplish in terms of metrology. By virtue of the formulation according to the invention, the turbidity of the solubilizate is preferably less than 50 FNU, more preferably less than 25 FNU, and most preferably less than 15 FNU, as measured by scattered light measurement with infrared light according to the provisions of the ISO 7027 standard at a dilution of the solubilizate in a ratio of 1:50 in water. The aqueous dilution of the solubilizate in a ratio of 1:50 has a pH in the range from 6 to 8.

For the experimental determination of the turbidity of the solubilizates according to the invention, the turbidity meters are calibrated with a standard suspension. The reading is therefore not provided in the form of measured light intensity, but as the concentration of the calibration suspension. When measuring any suspension, the reading therefore means that the liquid in question causes the same light scattering as the standard suspension of the indicated concentration. The internationally established turbidity standard is formazine. One of the most common units is “FNU” which stands for Formazine Nephelometric Units”. This is the unit used in water treatment, for example, for measurement at 90° in accordance with the provisions of the EN ISO 7027 standard.

Regarding bioavailability in mammals, Applicant commissioned a study conducted by Charles River Laboratories Den Bosch BV on male and female Wistar Han rats. Among other things, a solubilizate according to the invention of CBD in the form of an isolate as a starting material was compared with a solution of CBD isolate in glycerol with regard to bioavailability.

During the study, the animals were kept at a temperature ranging from 18° C. to 24° C., with a mean of 20° C. Air humidity ranged from 40% to 70%, with a mean in a range from 48% to 40%. The light cycle was set to 12 hours of light and 12 hours of darkness. The animals were fed SM R/M-Z from SSNIFF® Spezialitaten GmbH, Soest, Germany in the form of pellets, as well as water from the municipal water supply. The animals were allowed to consume food and water ad libitum, except for the administration of the dosage of the active substance. For the latter purpose, food intake was stopped the night before the active substance was administered for a maximum duration of 20 hours, although water intake was still permitted. Food intake was allowed to be resumed 4 hours after administration of the dosage.

A dosage of 41.1 mg/kg body weight was administered orally once as a single dose to 10 animals of each test group. Blood samples with a volume of 0.2 mL in K₂EDTA as anticoagulant were collected via jugular vein puncture at time points 15 min (0.25 hours), 30 min (0.5 hours), 45 min (0.75 hours), 1, 2, 4, 8, and 24 hours after conclusion of dosing.

The samples were centrifuged within 2 hours at approximately 2000 g for 10 minutes at a temperature in the range from 4° C. to 8° C. The resulting plasma was separated and frozen in polypropylene tubes immediately over dry ice or in a freezer set to −75° C.

The parameters described in Table 1 were determined for the pharmacokinetics.

No mortality and no clinical signs of toxicity of the solubilizate were observed during the study.

Table 2 shows the results of the study on the basis of the parameters explained in Table 1. The mean values given for the specified AUC and tₘₐₓ and Cₘₐₓ are based on the data for the 10 animals in each group.

TABLE 1
Pharmacokinetic parameters

| Parameter | Description |
|---|---|
| tₘₐₓ | Point in time following the administration, at which |
| | the maximum concentration was observed |
| Cₘₐₓ | Maximum concentration measured following the |
| | administration |
| AUC | Area under the concentration-time curve |
| | from the onset of administration . . . |
| AUC_{(0-0.25 h)} | . . . until t = 0.25 h. |
| AUC_{(0-0.5 h)} | . . . until t = 0.5 h. |
| AUC_{(0-0.75 h)} | . . . until t = 0.75 h. |
| AUC_{(0-1 h)} | . . . until t = 1 h. |
| AUC_{(0-2 h)} | . . . until t = 2 h. |
| AUC_{(0-4 h)} | . . . until t = 4 h. |
| AUC_{(0-8 h)} | . . . until t = 8 h. |
| AUC_{(0-inf)} | . . . extrapolated to infinity. Determined from single |
| | dose data only, if applicable |

TABLE 2
Values from the study

| | | | Solubilizate of the | |
|---|---|---|---|---|
| | | | CBD isolate acc. | |
| | | | to the invention | |
| | | | | Factor for the |
| | | CBD isolate | | value of the |
| | | dissolved in | | solution in |
| | Unit | glycerol | | glycerol |
| AUC_{(0-0.25 h)} | (h * ng/mL) | 0.78 | 11.6 | 14.88 |
| AUC_{(0-0.5 h)} | (h * ng/mL) | 4.19 | 82.55 | 19.70 |
| AUC_{(0-0.75 h)} | (h * ng/mL) | 12.39 | 238.5 | 19.25 |
| AUC_{(0-1 h)} | (h * ng/mL) | 27.90 | 449 | 16.09 |
| AUC_{(0-2 h)} | (h * ng/mL) | 137.55 | 1435 | 10.43 |
| AUC_{(0-4 h)} | (h * ng/mL) | 393.50 | 3465 | 8.81 |
| AUC_{(0-8 h)} | (h * ng/mL) | 1455.00 | 6075 | 4.18 |
| AUC_{(0-inf)} | (h * ng/mL) | 2520.00 | 9310 | 3.69 |
| tₘₐₓ | h | 6.2 | 2.1 | 0.34 |
| Cₘₐₓ | ng/mL | 468.5 | 1141 | 2.44 |

For the solubilizate according to the invention, the ratio of the respective value of the solubilizate to the value of the CBD isolate dissolved in glycerol is given in the right-hand column of Table 2 as “Factor for the value of the solution in glycerol”. This analysis in particular reveals a significant benefit of the invention. Firstly, the maximum concentration achieved after administration of the dose is more than twice as high (Cₘₐₓ of the solubilizate/Cₘₐₓ of the solution=2.44).

However, it is not primarily the absolute value of Cₘₐₓ that is decisive within the context of the invention, but rather the kinetics of absorption and degradation of the cannabinoid, which is enabled by the formulation according to the invention. This kinetics can be influenced by varying the composition within the scope of the invention and is reflected in the temporal course of the AUC after administration of the solubilizate in a significantly increased rate of absorption compared to the solution of the cannabinoid in glycerol, especially within the first hour after administration of the active substance (“onset”).

The AUC (Area Under the Curve) of the plasma concentration-time curve measured for up to 30 minutes after oral administration of the solubilizate according to the invention has a value which is higher by a factor of 17 to 22, preferably by a factor of 19 to 20, than after oral administration of the same dose of cannabinoid, in particular cannabinoid isolate, dissolved in glycerol.

The AUC (Area Under the Curve) of the plasma concentration-time curve measured for up to 60 minutes after oral administration of the solubilizate according to the invention has a value which is higher by a factor of 14 to 19, preferably by a factor of 16 to 17, than after oral administration of the same dose of cannabinoid, in particular cannabinoid isolate, dissolved in glycerol.

Moreover, the highest concentration was reached about three times as quickly in the animal study, i.e. in about a third of the time (tₘₐₓ of the solution/tₘₐₓ of the solubilizate=2.95). Thus, within the scope of the invention, the time tₘₐₓ of the plasma concentration-time curve after oral administration of a dose of cannabinoid, in particular cannabinoid isolate, in glycerol, at which the maximum concentration in the plasma is reached, has a value in the range of 1.96 times to 4 times, preferably in the range of 2.8 times to 3.1 times, of the value after oral administration of the same dose of cannabinoid, in particular cannabinoid isolate, in the form of the solubilizate according to the invention.

In particular, the invention thus provides for the use of a solubilizate as described above as a medication, in particular in the treatment of and/or prevention of sleep disorders (insomnia), depression and/or anxiety states. After taking a dose of the solubilizate, the highest concentration of the cannabinoid is reached within the first hour, thus providing for a rapid effect, in particular a fast deep sleep. After the first hour, the concentration of the active substance decreases again, so that a formulation is provided which allows degradation within a sleep period of up to 8 hours, for example. Thus, side effects such as daytime fatigue can be avoided by the invention.

The solubilizate according to the invention is therefore suitable for use as a dietary supplement and/or as a medicinal product with an effect against sleep disorders (insomnia), depression and/or anxiety. It also provides for the manufacture of a dietary supplement and/or a medicinal product for the treatment and/or prevention of sleep disorders (insomnia), depression and/or anxiety.

The significantly improved kinetics with regard to the bioavailability of the cannabinoid in the solubilizate according to the invention compared to the form dissolved in glycerol allows for a reduction in the amount of cannabinoid, in particular CBD, that a user has to take orally every day in order to achieve the desired effect.

In order to enable the oral application of the solubilizate according to the invention in a simple and convenient manner for the consumer or patient, the invention also provides a capsule filled with a solubilizate as described above, which capsule comes in the form of a soft gelatin capsule or hard gelatin capsule, or as a soft gelatin-free capsule or as a hard gelatin-free capsule, e.g. a cellulose capsule.

Within the scope of the invention, the solubilizate according to the invention can also be incorporated into other products, particularly fluids, in a particularly simple manner into liquids. In this process, the small micelles filled with the active substance remain intact. The fluids can be further processed so that the solubilizate can be used in any formulated product. The “product” within the meaning of the invention encompasses fluids and solids, including products for sucking by the consumer, such as coated tablets or gummy candies.

Thus, the invention also provides a product comprising a solubilizate as described above, wherein the product is selected from the group containing foodstuffs, dietary supplements, beverages, cosmetics, and pharmaceutical products. In particular, the product may comprise an aqueous dilution of the solubilizate within the scope of the invention.

The invention furthermore provides a method for treating and/or preventing sleep disorders (insomnia), depression and/or anxiety, in which a solubilizate according to the invention, in particular in a capsule or in the form of a fluid, is administered to the dietary supplement consumer or patient, in particular orally, in particular once a day.

It has been found to be already sufficient if the solubilizate is administered to the consumer or patient in a cannabinoid dose in the range from 5 mg/kg body weight to 8.4 mg/kg body weight, preferably in a dose of 6.7 mg/kg body weight, in particular once a day. The findings on dosage obtained in the animal study were converted by a factor of

$$dosage for humans / dosage for rats = 0.16 .$$

For a person weighing 75 kg, the dosage corresponds to a dose of 500 mg of cannabinoid, for example CBD.

The invention furthermore provides a method for producing a solubilizate as described above, comprising the following steps:
(a) providing at least one emulsifier having an HLB value in the range of less than 18, preferably between 13 and 18, in particular polysorbate 80 or polysorbate 20, or at least one sugar ester of edible fatty acids, or a mixture of at least two emulsifiers, selected from the group consisting of polysorbate 20, polysorbate 80, and sugar esters of edible fatty acids,
(b) mixing the emulsifier from step (a) with at least one cannabinoid, in particular cannabinoid isolate, and with at least one medium-chain triglyceride (MCT),
   wherein at least step (b) includes heating to a temperature in the range from 82° C. to 97° C., preferably to a temperature in the range from 83° C. to 92° C., most preferably to a temperature in the range from 85° C. to 89° C.

This already allows to produce a solubilizate according to the invention in a surprisingly simple manner. This preparation method enables the creation of a solubilizate which, when diluted in water, can form micelles that are loaded with at least one cannabinoid as an active substance.

Depending on the application and the specific intended formulation for the solubilizate, step (b) may also comprise adding the at least one cannabinoid together with other components. More particularly, premixes of the cannabinoid with at least one other component of the solubilizate to be produced may be prepared and then further processed with the emulsifier and, optionally, additional components.

A refinement of the method comprises, prior to step (b), a step
(b1) of mixing the at least one cannabinoid, in particular cannabinoid isolate, with the at least one medium-chain triglyceride (MCT).

Depending on the desired loading and particle size distribution, separate premixing of cannabinoid and MCT may be preferable.

Furthermore, it was found to be advantageous for the preparation of a mixture which is as uniform as possible, if step (a) includes heating to a temperature at least in the range from 40° C. to 62° C., preferably to a temperature in the range from 45° C. to 57° C., most preferably to a temperature in the range from 48° C. to 52° C.

In a further embodiment of the invention, glycerol is additionally added to the mixture in step (b) and/or in step (b1). It is also possible within the scope of the invention to first prepare a premix of glycerol and cannabinoid. For this purpose, a step
(b11) of mixing at least one cannabinoid, in particular cannabinoid isolate, with glycerol may be carried out prior to step (b) and/or prior to step (b1).

By adding glycerol, the stability of the micelles of the solubilizate can be improved, if needed. Depending on the application case and production conditions, it may be helpful to protect the cannabinoid from decomposition, in particular if it is processed as an isolate. For this purpose, it is contemplated according to a refinement of the invention to additionally add at least one antioxidant, preferably tocopherol, most preferably mixed tocopherol, in step (a).

The invention will now be discussed in more detail by way of an exemplary embodiment. The following components were used:

### Polysorbate 80

The material “TEGO SMO 80 V FOOD” from Evonik Nutrition & Care GmbH, Essen, Germany, was used as the source of polysorbate 80. This product complies with EU requirements for the food additive E 433. Alternatively, the product Crillet 4/Tween 80-LQ-(SG) from CRODA GmbH, Nettetal, Germany, can be used.

In addition or as an alternative to polysorbate 80, it would also be possible within the scope of the invention to use polysorbate 20 and/or sugar esters of edible fatty acids as an emulsifier. One possible source of polysorbate 20 is the material “TEGO SML 20 V FOOD” with the specification code “K09 EU-FOOD” from Evonik Nutrition & Care GmbH, Essen, Germany, for example. This product complies with the EU requirements for food additive E 432. As an alternative to the aforementioned TEGO SML 20 from Evonik, it is also possible to use Crillet 1/Tween 20-LQ-(SG) from CRODA GmbH, Nettetal, Germany as polysorbate 20 within the scope of the invention. One example of a possible sugar ester of edible fatty acids is a sucrose ester with the product name “DUB SE 15 P” from the manufacturer Stdarine Dubois. This is approved in the EU as food additive E 473.

### Glycerol

The glycerol used in the context of the present application was the product “Glycerol 99.5%” from Cremer Oleo GmbH & Co. KG, Hamburg, Germany. Alternatively, the product “Glycamed 99.7%” from Glaconchemie GmbH, Merseburg, Germany, can be used. According to the manufacturer, the glycerol content of this product is at least 99.5%.

### Medium-Chain Triglycerides

Medium-chain triglycerides were used in the form of MCT oil (70/30) Rofetan GTCC 70/30 manufactured by DHW Deutsche Hydrierwerke Rodleben GmbH, Dessau-Roßlau, Germany. Alternatively, the product Delios S from BTC Europe GmbH, Monheim, Germany, can also be used, for example.

### Mixed Tocopherol

As a mixed tocopherol (E306, CAS numbers 59-02-9, 16698-35-4, 54-28-4, and 119-13-1), the 70% mixed tocopherol in vegetable oil Vitapherole T-70 Non GMO manufactured by VitaeNaturals can be used, for example.

### EXEMPLARY EMBODIMENT: CBD SOLUBILIZATE

The following is used:

| 33 g | CBD isolate, cannabidiol at least 99.8% |
|---|---|
| (32.9 g CBD) | |
| 40 g | glycerol, |
| 40 g | MCT oil, |
| 15 g | mixed tocopherol 70%, and |
| 872 g | polysorbate 80. |

The CBD isolate is cannabidiol of CAS number 13956-29-1, which comes in the form of a whitish to slightly yellowish powder.

Polysorbate 80 is heated to a temperature in the range from 48° C. to 52° C. Mixed tocopherol is added and homogenized with stirring while maintaining the temperature. Stirring is performed vigorously enough to ensure an even distribution of the mixed tocopherol in the polysorbate 80. Glycerol, MCT oil, and the CBD isolate are mixed separately and heated to a temperature of 85° C. to 89° C. and are thereby homogenized completely so that the turbidity of this pre-solution no longer changes with further stirring while maintaining the temperature. Subsequently, the mixture of glycerol, MCT oil, and CBD isolate is incorporated into the mixture consisting of polysorbate 80 and mixed tocopherol while stirring. Stirring is performed vigorously enough to ensure an even distribution while maintaining the temperature in the range of 85° C. to 89° C. The product is then cooled to a temperature below 60° C. and bottled. It is then stored in the dark at a maximum of 25° C.

When diluted 1:50 in water, this 3% CBD isolate solubilizate has a maximum turbidity of 50 FNU. A value of 10.4 was measured on the sample as the mean value from three measurements on samples from three batches, and thus a value of well below 15 FNU. It is particularly suitable for administration in capsules or as an additive to beverages.

In the formulation according to the invention and the exemplary embodiment described above, the mixed tocopherol serves as an antioxidant. Depending on the intended use or other components of the formulation of the product in which the solubilizate according to the invention is used, the addition of the mixed tocopherol may be dispensed with, mixed tocopherol may be used in combination with other substances, or another substance or mixture of substances may be used as an antioxidant.

Within the scope of the invention, in addition to CBD isolate, cannabinoid solubilizates may additionally or alternatively be produced and used with THC oil or THC isolate and/or with oils or isolates or other sources of other cannabinoids as well. The advantage of using an isolate compared to using a cannabinoid oil for the formulation of the solubilizate is that, due to the high concentration of active substance in the isolate, a comparatively low mass of this component containing the active substance can be used in the solubilizate, while still achieving a high cannabinoid content in the solubilizate.

It will be apparent to those skilled in the art that the invention is not limited to the examples described above, but rather can be varied in various ways. In particular, the features of the individual examples presented can also be combined with one another or interchanged. This applies in particular to the emulsifier composition and the active substance composition of the solubilizate according to the invention.

1. A solubilizate, comprising
at least one cannabinoid, in particular a cannabinoid isolate;
at least one emulsifier having an HLB value in the range of less than 18, preferably between 13 and 18, in particular polysorbate 80 or polysorbate 20, or at least one sugar ester of edible fatty acids, or a mixture of at least two emulsifiers, selected from the group comprising polysorbate 20, polysorbate 80 and sugar esters of edible fatty acids; and
medium-chain triglycerides, wherein the solubilizate includes medium-chain triglycerides in a proportion of up to 10 wt %, preferably between 1 wt % and 8 wt %, most preferably up to 5 wt %.

2. The solubilizate according to claim 1, wherein the solubilizate contains, as a further component, up to 10 wt % of glycerol.

3. The solubilizate according to claim 1, wherein the solubilizate contains, as a further component, up to 5 wt % of at least one antioxidant.

4. The solubilizate according to claim 1, wherein the emulsifier content is at least 70 wt %.

5. The solubilizate according to claim 1, wherein the diameter distribution of the micelles in a dilution of the solubilizate with distilled water in a ratio of 1:500 under physiological conditions (pH 1.1 and 37° C.) ranges from about d₁₀=5 nm to about d₉₀=20 nm, preferably from d₁₀=6 nm to about d₉₀=13 nm.

6. The solubilizate according to claim 1, wherein the turbidity of the solubilizate is less than 50 FNU, preferably less than 25 FNU, most preferably less than 15 FNU, as measured by scattered light measurement with infrared light according to the provisions of the ISO 7027 standard at a dilution of the solubilizate in a ratio of 1:50 in water.

7. The solubilizate according to claim 1, wherein the AUC (area under the curve) of the plasma concentration-time curve measured for up to 30 minutes after oral administration of a solubilizate according to any one of the preceding claims has a value which is higher by a factor of 17 to 22 than after oral administration of the same dose of cannabinoid, in particular cannabinoid isolate, dissolved in glycerol.

8. The solubilizate according to claim 1, wherein the AUC (area under the curve) of the plasma concentration-time curve measured for up to 60 minutes after oral administration of a solubilizate according to any one of the preceding claims has a value which is higher by a factor of 14 to 19 than after oral administration of the same dose of cannabinoid, in particular cannabinoid isolate, dissolved in glycerol.

9. The solubilizate according to claim 1, wherein the time tₘₐₓ of the plasma concentration-time curve after oral administration of a dose of cannabinoid, in particular cannabinoid isolate, dissolved in glycerol, at which the maximum concentration in the plasma is reached, has a value in the range of 1.96 times to 4 times the value after oral administration of the same dose of cannabinoid, in particular cannabinoid isolate, in a solubilizate according to any one of the preceding claims.

10. (canceled)

11. (canceled)

12. (canceled)

13. A capsule filled with a solubilizate according to claim 1, wherein the capsule is in the form of a soft gelatin capsule or hard gelatin capsule, or a soft gelatin-free capsule, or a hard gelatin-free capsule, for example a cellulose capsule.

14. A product comprising a solubilizate according to claim 1, wherein the product is selected from the group containing foodstuffs, dietary supplements, beverages, cosmetics, and pharmaceutical products.

15. The product of claim 14, wherein the product comprises an aqueous dilution of the solubilizate.

16. A method for treating and/or preventing sleep disorders (insomnia), depression and/or anxiety, wherein a solubilizate according to claim 1 is administered to a dietary supplement consumer or patient, in particular orally.

17. The method of claim 16, wherein the solubilizate is administered to the dietary supplement consumer or patient in a cannabinoid dose in a range from 5 mg/kg body weight to 8.4 mg/kg body weight.

18. A method for producing a solubilizate according to claim 1, comprising the steps of:
(a) providing at least one emulsifier having an HLB value in the range of less than 18, or at least one sugar ester of edible fatty acids, or a mixture of at least two emulsifiers, selected from the group consisting of polysorbate 20, polysorbate 80, and sugar esters of edible fatty acids;
(b) mixing the emulsifier from step (a) with at least one cannabinoid, in particular cannabinoid isolate, and at least one medium-chain triglyceride (MCT),
wherein the medium-chain triglyceride is used in a content of up to 10 wt.-%;
wherein at least step (b) includes heating to a temperature in the range from 82° C. to 97° C.

19. The method of claim 18,
wherein prior to step (b), a step (b1) of mixing at least one cannabinoid, in particular cannabinoid isolate, with at least one medium-chain triglyceride (MCT) is carried out.

20. The method of claim 18, wherein in step (a) heating to a temperature at least in the range from 40° C. is carried out.

21. The method of claim 18, wherein in step (b) and/or step (b1) additionally glycerol is added.

22. The method of claim 18, wherein prior to step (b) and/or prior to step (b1), a step (b11) of mixing at least one cannabinoid, in particular cannabinoid isolate, with glycerol is carried out.

23. The method of claim 18, wherein in step (a) additionally at least one antioxidant is added.
